(19) United States
(12) Patent Application Publication
(10) Pub. No.: US 2026/0232337 A1
(43) Pub. Date: Aug. 13, 2026

(54) MULTI-FUNCTION THROMBECTOMY HUB

(71) Applicant: Vetex Medical Limited, Galway (IE)

(72) Inventors: Morgan Tierney, Co Offaly (IE); Adrian Mahon, Co Offaly (IE); Anthony Wright, Waterford (IE); Eugene Skelton, Dublin (IE)

(21) Appl. No.: 19/160,824

(22) PCT Filed: Mar. 1, 2024
(86) PCT No.: PCT/EP2024/055504

§ 371 Date: Aug. 28, 2025

Related U.S. Application Data
(60) Provisional application No. 63/449,711, filed on Mar. 3, 2023.

(51) Int. Cl. A61B 17/221; A61B 17/00; A61B 17/3207

(52) U.S. Cl. CPC A61B 17/221; A61B 17/00234; A61B 17/320758; A61B 2017/00305; A61B 2017/00367; A61B 2017/00778; A61B 2017/2212

(57) ABSTRACT

A catheter system (100) includes a catheter shaft (115) having an outer catheter shaft (130) within an outer sheath (120). An inner catheter shaft (140) is movably coupled within the outer catheter shaft. A cage (150) is proximate to a distal end of the catheter shaft, and a cage distal portion is coupled with the inner catheter shaft and a cage proximal portion is coupled with the outer catheter shaft. A hub assembly is coupled with the catheter shaft and includes a control interface (20) movably coupled with a hub body (210). The control interface is configured to actuate the outer sheath and the cage.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever. The following notice applies to the software and data as described below and in the drawings that form a part of this document: Copyright Vetex Medical of Galway, Ireland. All Rights Reserved.

### TECHNICAL FIELD

This document pertains generally, but not by way of limitation, to compact catheter systems with a control system including a movable interface where the movable interface is configured to translate movement to multiple components of the catheter system.

### BACKGROUND

A surgical device, such as a catheter, provides access to surgical sites in a body such as through an opening, cavity or tract. In certain medical procedures, an opening is provided through an incision made in the body and a catheter shaft is inserted in the incision or opening. The shaft is navigated through vasculature to a target location.

In certain medical procedures, the catheter shaft is passed through the body to a target location; however, advancing the catheter shaft to the target location is in some examples difficult because of tortuous and irregular vasculature. In some examples, a catheter shaft decreases challenges of reaching a target location. In one example, the shaft includes smooth exterior surfaces, and one or more working components are received in the lumen of the catheter shaft and delivered from a distal opening of the catheter shaft.

In other examples, a sheath or covering is provided that at least partially covers one or more working components, for instance extending from a catheter shaft distal opening. In some examples the working components include thrombectomy devices configured to capture, macerate or extract thrombus. The sheath covers the working components during delivery and is retracted proximally to expose the working components.

In some examples, the sheath and working components are coupled with control interfaces of a surgical device. Optionally, the sheath and working components are coupled to separate control interfaces that are moved relative to each other, for instance by cooperating technicians or doctors. The control interfaces permit doctors and technicians to cooperatively control the surgical device at a distal end of the catheter shaft. In an example, the hub can be sized to house, contain or the like, mechanism, such as motors or control mechanisms, which are used to control the working components.

### SUMMARY

The present inventors have recognized, among other things, a problem exists related to systems for containing or holding all or the majority of the operational components for certain surgical systems, such as catheters. Such a problem includes how to house or contain the operating system contained within a single hub. However, if such an operating system exists, they are so cumbersome or bulky it is not accurate to just say the operating system is a hub because it is more akin to a system manipulation hub or peripheral unit. Such a large system requires, for example, multiple doctors or technicians to work the system. A large system also requires the operating system to be placed on a surface, such as a surgical table, side table or tray during use because of the system's bulk.

The present inventors have recognized a way for solving the problem related to the bulkiness of the hub or peripheral unit can include combining operating components into a single, easy to use control interface. A single control interface, for example, minimizes elements used for transferring movement to external components. Minimizing the real estate required to transfer movement results, in examples, in a control system sized to be operated by one medical professional. The medical professional, in such an example, is able to hold the control system in one hand similar to a handle or remote control.

A control system sized to be held and operated in an adult's hand in an example, includes control interfaces operatable by one digit, such as the thumb. The present inventors have recognized an advantage in the control interface being operatable by movement in directions according to the dexterity of a human thumb or the like. This movement by a thumb or digit on the control interface, such as moving a rotating dial, a slide, or joystick, is selectively couplable via clutches or actuators within the control system, to translate movement to shafts, sheathes or other external features of a catheter system. For example, the control interface of a catheter system for removing a clot is configured to selectively actuate at least one of an outer sheath, and inner shaft, and a cage. The actuation of each of the outer sheath, inner shaft and cage is controlled, for example, by a control interface configured to be moved with a first movement and optionally a second movement with the first and second movements, for example, being collinear (e.g., aligned, parallel, in a similar direction or the like), and in one example being in a common direction (e.g., proximal, distal, transverse, oblique or the like).

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope of the present invention is defined by the appended claims and their legal equivalents.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example of a cross section of catheter system according to at least one example of the application.

FIG. 2A illustrates an example of a cross section of a hub of a catheter system according to at least one example of the application.

FIG. 2B illustrates an example of a hub of a catheter system according to at least one example of the application.

FIG. 3 illustrates an example of a control interface of a catheter system according to at least one example of the application.

FIG. 4 illustrates an example of a control interface of a catheter system according to at least one example of the application.

FIGS. 5A-5D illustrates cross sections of a catheter system according to at least one example of the application.

### DETAILED DESCRIPTION

Catheter assemblies are an example of a device suitable for use in a body lumen comprising an elongated member suitable for insertion into the human body and a control mechanism external of the human body. In an example, a catheter system used for thrombectomies is an example of an extraction device that has a radially expansible member such as a cage, basket, funnel, ring or the like (hereinafter “cage”). The cage is disposed at, or proximate to, a distal end of the elongated member. In an example, the cage is adapted to be adjustable between a contracted orientation and an expanded orientation. The contracted orientation is, for example, suitable for insertion and advancing though body lumens to a target site and removal from the body lumen and out of the human body. Conversely, the cage is also adapted to take an expanded orientation. For example, in the expanded orientation, the cage is configured to collect the matter (i.e., a thrombus or other blockage) and remove matter from the interior of the body lumen.

In an example, the catheter system has a plurality of arms (such as shafts, sheaths, tubes, or the like) at least one of which is connected at a distal portion adjacent to a proximal end of the cage and another arm is connected to or adjacent to a distal end of the cage. A proximal end of the plurality of arms is connected to a control mechanism that is the interface for a medical professional. The control mechanism, for example, controls movement of one of the arms relative to the other results in adjustments of the diameter or radial strength of the cage. For example, moving one arm relative to the other translates to adjustments of the diameter from a contracted orientation to an expanded (deployed) orientation.

The control mechanism includes a connection with at least one of the plurality of arms. For example, the control mechanism includes a hub with a control interface. The control interface is optionally movably connected with at least one of the plurality of arms. In an example, each of the plurality of arms is configured to move at least one component of the catheter system. For example, the cooperation between the control interface with the plurality of arms can expand or contract the cage.

The plurality of arms is configured for use in tapering and tortuous lumens with obstructions, such as valves, where movement of the cage in a deployed, or expanded, shape along the lumen requires the diameter of the cage to change. For example, when the lumen is tapering inwardly (in the direction of travel of the cage), the control mechanism is configured to exert a force on the cage (by biasing the cage into an expanded configuration or by holding at least one of the plurality of arms in a specific orientation or by rotating the cage while in an expanded or partially expanded configuration) until the force exerted on the cage by the lumen exceeds the total resistance force including that force exerted by the resistance mechanism at which the diameter of the cage will reduce. This configuration, for example, provides a configuration for the cage to be moved along the walls of the lumen while exerting the radial force against the lumen walls to affect a scraping/collecting action.

In an example, the cage is made from a braid, a series of wires, laser cut tubes or a combination or any similar means which can be easily deformed during use. The cage is made, for example, from polymeric metal such as stainless-steel Nitinol or cobalt chromium or ceramic, or any similar material. In one embodiment, the cage is made from a combination of a material and design which is configured to be easily collapsible and expandible. In an example when the cage is in the expanded configuration, the cage is also configured and formed to act as a filter, structure, or passageway to collect a thrombus.

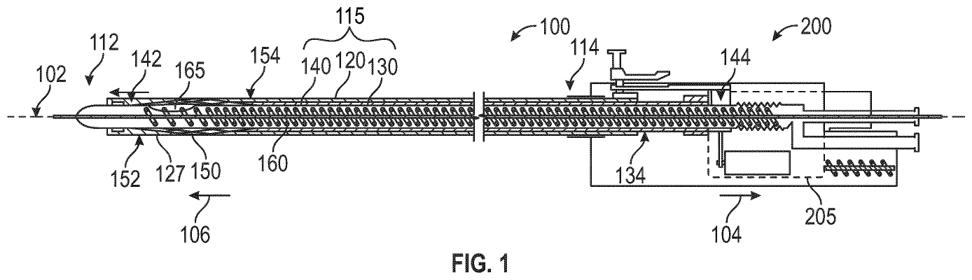

FIG. 1 illustrates an example of a catheter system 100 with a plurality of shafts 115. The catheter system 100 is an example of an extraction device. In one configuration, at least one of the plurality of shafts 115 is coupled with a cage 150 proximate to a distal end 112 of the plurality of shafts 115 and a control mechanism 200 at a proximal end of the plurality of shafts 115. In an example, the expansion and contraction of the cage 150 is operated and controlled by cooperation between the control mechanism 200 and the plurality of shafts 115 of the catheter system 100. For example, at least one of the plurality of shafts 115 is coupled with the cage 150. In an example, at least one of the plurality of shafts 115, such as shaft 140, is coupled with the cage 150 proximate to a distal portion 152 of the cage 150. In an example, at least one of the plurality of shafts 115, such as shaft 130, is coupled with the cage 150 at a proximal portion 154 of the cage 150.

In an example, the plurality of shafts 115 includes at least three colinear shafts, such as shafts that share a central, longitudinally extending axis 102 (longitudinal axis 102). In an embodiment, from outside of the plurality of shafts 115 inward, there is an outer sheath 120, an outer shaft 130 and an inner shaft 140. The outer sheath 120, for example, covers, encases, or surrounds the outer shaft 130. The outer shaft 130, in an example, covers, encases, or surrounds the inner shaft 140. In an embodiment, the inner shaft 140 at least partially covers, encases, or surrounds an extractor 160. The inner shaft 140 is configured for, in one example, drawing in of thrombus and passage of thrombus that has been gathered or filtered by the cage 150 and passed through the cage 150. Thrombus, for example, is drawn into the inner shaft 140 and comes in contact with the extractor 160. The extractor 160 breaks the thrombus down, such as by maceration, pulverizing, subdividing, partitioning, squeezing or the like to fragmentize the thrombus.

The extractor 160 includes, but is not limited to, a rotatable mechanism such as a coil, auger, or the like that is configured to deliver the fragmentized thrombus proximally through the inner shaft 140 to an exit from the catheter system 100. The extractor 160 is exposed, for example, via a window or opening 165 (hereinafter “window”) in the inner shaft 140. The window 165, in one configuration, is formed within the inner shaft 140 in an area at least partially surrounded by the cage 150.

As shown in the example illustrated in FIG. 1, the outer sheath 120 extends over at least a portion of the cage 150 with the cage 150 in a contracted or sheathed configuration (hereinafter “sheathed configuration”). For example, in a sheathed configuration, the cage 150 is covered or enveloped by the outer sheath with the cage in a contracted or collapsed configuration, or the like. In an example, the outer sheath 120 extends over the cage 150 (including a portion or the entirety of the cage) with the cage 150 contracted (e.g., compressed, collapsed, reduced in size or the like). For example, the outer sheath 120 retains, holds, covers, encases or the like (hereinafter “covers”) the collapsed caged 150. The outer sheath 120 is made from one or more materials suitable for covering the cage 150 during insertion or removal of the plurality of shafts 115 from the body lumen. For example, the outer sheath 120 is made from one or more materials having a substantially smooth outer surface to facilitate passage or sliding of the sheath 120 through body lumens to a target site. In another example, the outer sheath 120 includes one or more pliable materials that permit deformation of the outer sheath 120 as it slides over the cage 150, optionally filled with thrombus material and partially collapsed. In an example, the material the outer sheath 120 is made from any one or more rigid materials that permits deformation of the outer sheath 120 as it slides over the cage 150 optionally filled with thrombus material.

In an example, at the proximal portion 114 of the outer sheath 120 there is a locking mechanism configured to retain the outer sheath 120 in place when the outer sheath 120 has been moved in a proximal direction. For instance, the locking mechanism is a pair of two or more components that engage with each other such as a detent (e.g., notch, recess, opening or the like) with a corresponding protrusion, a spring-loaded feature with a corresponding recess, a clip with a corresponding detent (e.g., notch, recess, opening or the like). In an example, the locking mechanism is unlocked (e.g., disengaged) from a locking configuration by the application of a reverse force, for instance provided with the control interface 220.

As illustrated in FIG. 1, the outer shaft 130 is an example of one of the plurality of shafts 115 and is retained within the outer sheath 120. In an example, the outer shaft 130 extends from within the control mechanism 200, at a proximal portion 134 of the outer shaft 130, to the distal portion 152 of the cage 150. The proximal portion 134 of the outer shaft 130, in an example, is configured to be substantially stagnant or fixed within the control mechanism 200. The outer shaft 130, for example, retains the inner shaft 140 to prevent the inner shaft 140 from becoming misaligned during use.

In an example, the inner shaft 140 is an elongate, hollow shaft retained within the outer shaft 130. For example, a proximal portion 144 of the inner shaft 140 is coupled, held, retained, or the like, within the control mechanism 200 and a distal portion 142 of the inner shaft is configured to be coupled with the cage 150 at a distal portion 152 of the cage 150.

In an example, the inner shaft 140 is movably coupled with respect to the remaining of plurality of shafts 115. For example, the inner shaft 140 is slidably coupled within the outer shaft 130. The outer shaft 130, for example, remains substantially static (e.g., fixed or minimal movement) relative to the inner shaft 140. In another example, the inner shaft 140 is translatable from a first position to at least a second position, and to further positions if dictated by the design. The inner shaft 140 is movably coupled within the control mechanism 200, such as withdrawn in a proximal direction 104 or extended in a distal direction 106. In an example, the inner shaft 140 is withdrawn in the proximal direction 104 while remaining contained within at least a portion of the outer shaft 130.

At least one of the plurality of shafts 115, for example at least one of the outer sheath 120, outer shaft 130 and inner shaft 140, is coupled with at least one working part of the working components of the catheter system 100, such as the cage 150. The proximal portions 114, 134 or 144 of the plurality of shafts are selectively coupled with respective engagement components, such as clutches, of the control mechanism 200.

In examples of some catheter control systems, the control hub is bulky or cumbersome and therefore is difficult for one medical professional to operate easily. For example, the large and cumbersome control hub optionally requires a medical professional to rest the control hub on a surface during use. The size of the control hub can require a medical professional to use two hands to operate the control mechanisms on the control hub.

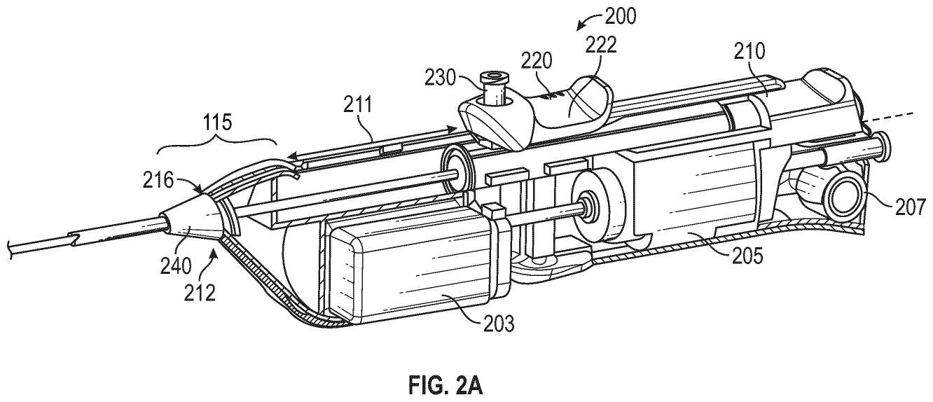

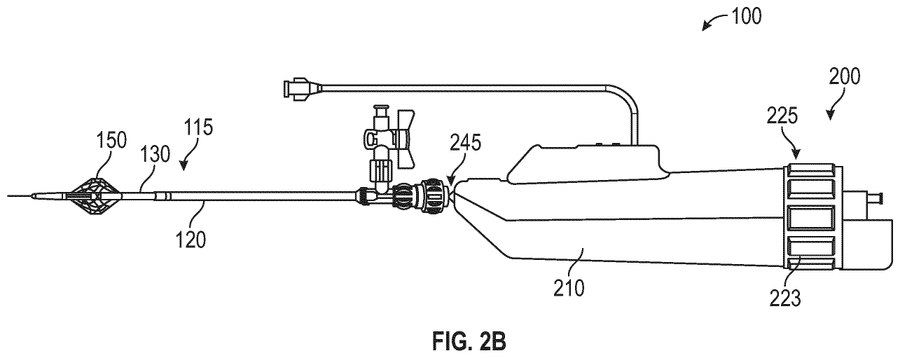

As illustrated in FIGS. 2A and 2B, the control mechanism 200 has a profile configured for operation by a medical professional with a single hand instead of being placed on a table or surface for operation or having a second medical professional assist in operation of the catheter system 100. As an example, the control mechanism 200 is sized similar to a remote control or handle. For example, the control mechanism 200 includes a housing or hub 210 having a hollow interior sized to house, contain, retain, or the like (hereinafter “house”) any required components for operation of the external components of the catheter system 100. For example, electromechanical, electronic and mechanical components are housed in the hub 210, as will be discussed below.

Referring to the example control mechanism of FIG. 2A, the cage 150 and the plurality of shafts 115 are configured to operate in cooperation with components housed in the hub 210 configured to translate movement to each of the plurality of shafts 115. For example, as illustrated in FIGS. 2A and 2B, on an external surface of the hub 210 there is a control interface 220, 225. The control interface 220, 225 is at least one of a slider, thumb slider, rotatable wheel, joystick, buttons, touch screen, sensor or the like. In one example, the control interface 220 is on an upper surface 211 of the hub 210. In another example, the control interface 220, 225 is placed in or on one or more locations on the hub 210, such as on the proximal end of the hub 210 as a rotatable control interface 225, as illustrated in FIG. 2B. The control interface 220, 225 is an example of a mechanism that controls at least one of the plurality of shafts 115, as discussed further below. The control interface 220, 225 is an example of a control mechanism that manipulates the cage 150, for instance to rotate, deploy (release or expand), contract the cage or the like.

In an example, the control interface 220, 225 has an engageable surface 222, 223 that a medical professional contacts to activate movement of the control interface 220, 225. The engageable surface 222, illustrated in FIG. 2A, includes a surface with a profile that accommodates the digit of a medical professional, such as a thumb. In another example, the engageable surface 223 includes surfaces of the rotatable control interface 225, such as a rotatable wheel, dial, or the like, as illustrated in FIG. 2B. The profile of the engageable surface 222, 223, in one example, decreases slipping of a digit such as a thumb, finger or the like. In an example, the engageable surface 222, 223 has a smooth or flat surface, optionally contoured in a complementary manner to a digit. In another example, the engageable surface 222, 223 has a knurled contour, such as a roughened, ridged, textured or irregular contour.

The control interface 220, 225 is manipulated (e.g., moved, rotated, pressed, displaced or translated) from at least one point or position to at least a second point or position and, optionally, to further points or positions therebetween. In an example, the movement of the control interface 220, 225 is a single movement. In another example the movement is a series of movements that are in a common direction. In an example, the control interface 220, 225 is shifted from one position to another. For example, the common direction is a direction for colinear movement from a first point or position to a second point or position and, optionally, further points or positions. The movement of the control interface 220 is, for example, in a proximal/distal direction relative to the catheter system 100. For example, the movement of the control interface 220 is in a proximal/distal direction that is also substantially parallel to a central axis of the control mechanism 200. The movement of the control interface 220, in another example, is in a direction askew or transverse from the direction of the central axis of the control mechanism 200.

In an example, the movement of the rotatable control interface 225 is in direction transverse, oblique, or the like relative to the longitudinal axis 102. For example, the movement of the rotatable control interface 225 includes a circular, arcuate or elliptical movement about the longitudinal axis 102. For example, the movement of the rotatable control interface 225 is optionally clockwise or counterclockwise according to the purpose (e.g., expansion or contraction of the cage 150, rotation of the shafts, or the like). The movement of the control interface 220, 225 in another example, is in any direction suitable for controlling, operation or manipulating the plurality of shafts 115 or the cage 150 at the distal end 112 of the plurality of shafts 115, as illustrated in FIG. 1.

For example, the control interface 220 is moved longitudinally from a first point to a second point and any further points. In another example, the control interface 220 is moved longitudinally to a first point or position and laterally in a second point or position. In another example, the control interface 220 is moved longitudinally in to a first point or position and vertically to a second point or position. In yet another example, the control interface 220, 225 moves in any combination of directions, as dictated by the purpose. For example, the control interface 220, 225 is moved relative to the longitudinal axis 102, either along the axis or around the longitudinal axis 102, to extend or retract any of the plurality of shafts 115. Hereinafter, control interface 220 will refer to control interface 220 as illustrated in FIG. 2A and rotatable control interface 225 as illustrated in FIG. 2B. In an embodiment, the control interface 220 includes features associated with operations of the catheter system 100 illustrated in FIG. 1. For example, the control interface 220 is configured to accommodate ports used during operation of the catheter system 100. One example port is a flush port 230 used to provide fluids, such as a saline solution, from an external source to components within the catheter system. The flush port 230, in an embodiment, is coupled with the plurality of shafts 115. For example, the flush port 230 is coupled with the outer sheath 120 to supply fluids which assist in cleaning the components of the catheter system 100.

As illustrated in FIG. 2A, the plurality of shafts 115 pass through an opening 216 at the distal end 212 of the control mechanism 200. For example, the plurality of shafts 115 pass through a coupling 245 at the distal end 212 of the hub 210 through which the plurality of shafts 115 pass into the hub 210. In an example, a strain relief 240 is provided at the distal end 212 of the control mechanism 200. The strain relief 240 is coupled with the control mechanism 200 to cover the opening 216, and optionally the coupling 245. The strain relief 240, in an embodiment, is engaged with the plurality of shafts 115 as the plurality of shafts 115 enter into the control mechanism 200. In an example, the strain relief 240 supports the plurality of shafts 115 and minimizes deformation, such as kinking or buckling, of the plurality of shafts 115 during use by bracing the plurality of shafts 115. For example, stresses otherwise carried by the plurality of shafts 115 at the interface with the control mechanism 200 are transmitted and absorbed (at least partially) by the strain relief 240, for instance when deflecting the plurality of shafts 115, translating the shafts 115 in a proximal or distal direction (e.g., relative to each other, relative to vasculature or the like).

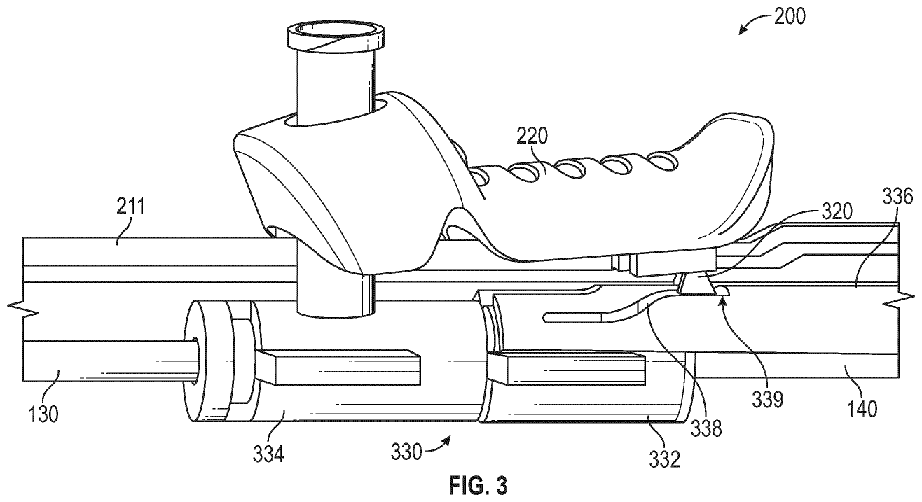

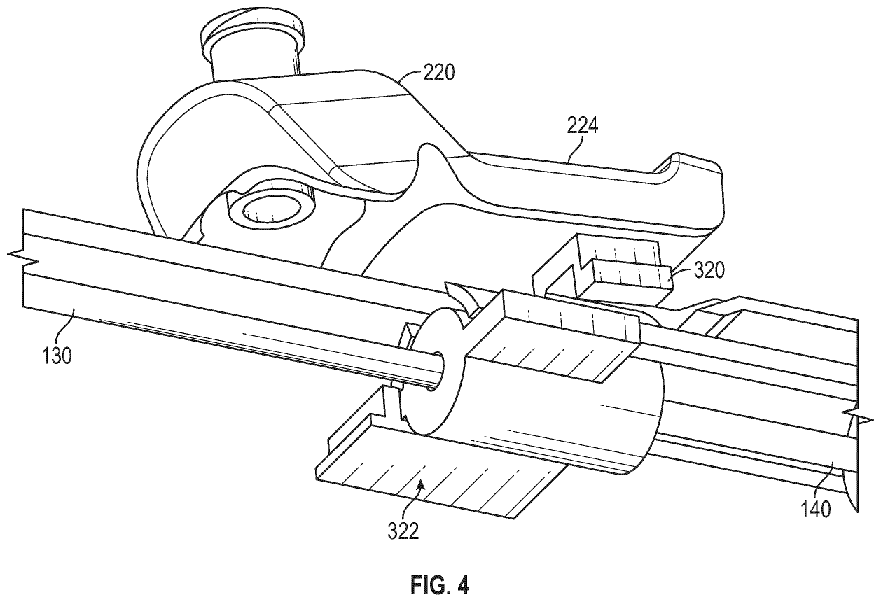

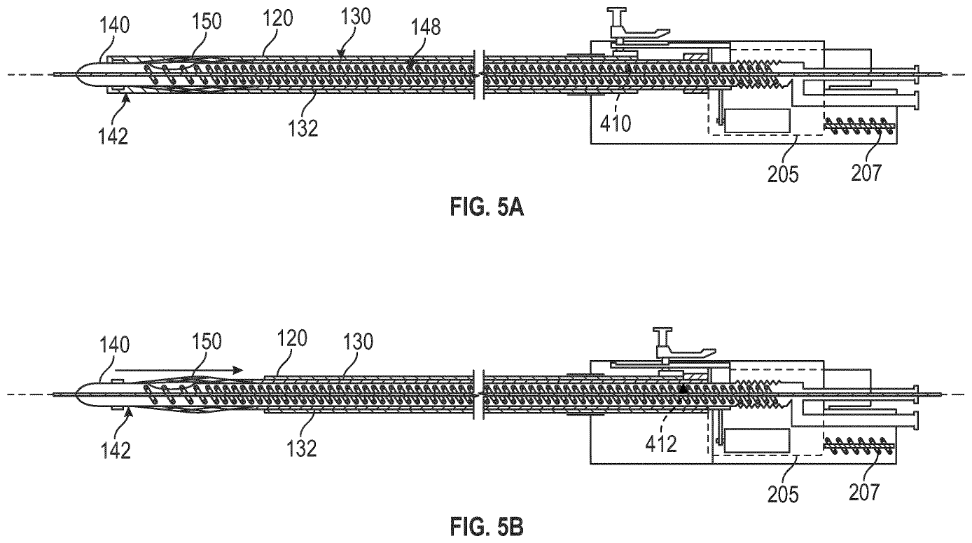

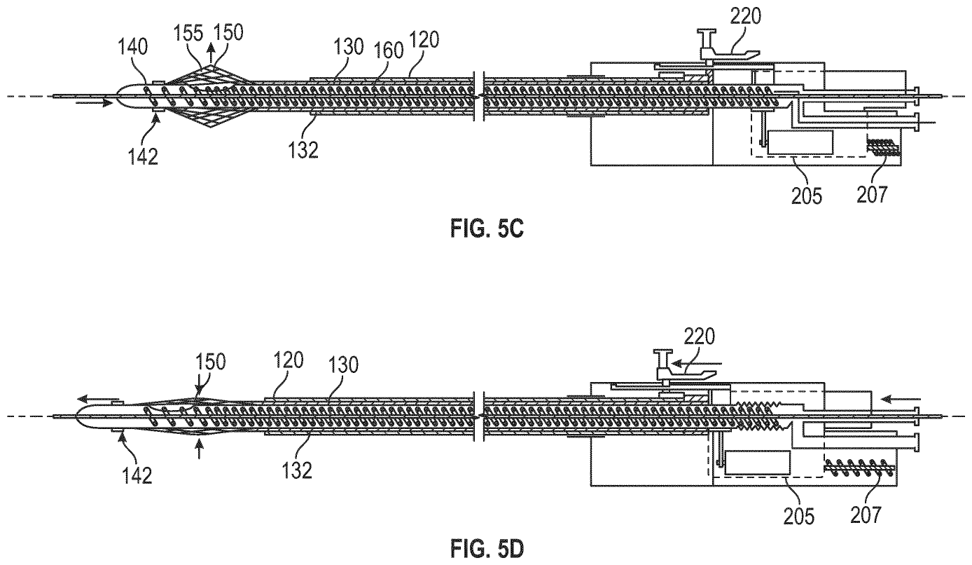

As illustrated in FIG. 3, the control mechanism 200 is configured to house clutches 330 selectively coupled to the plurality of shafts 115. For example, the clutches 330 include, but are not limited to, linkages, couplings, connections, shuttles and sockets or the like. The interaction between the control interface 220 and the plurality of clutches 330 are configured to actuate working components, such as the cage 150 coupled to the distal end 112 of at least one of the plurality of shafts 115. The cage 150, in an example, is an extension of at least one of the plurality of shafts 115. The cage 150, in another example, is housed within an end of at least one of the plurality of shafts 115. For example, the cage 150 is exposed with operation of the control mechanism 200 and the associated clutch (or clutches) upon reaching a target location, such as a location having blockage or a clot in the blood vessel, as will be discussed further below. Each of the plurality of shafts 115 (see FIG. 1) is coupled with at least one of the plurality of clutches 330 (such as outer catheter shaft Luer 322). In an example, there are clutches coupling each of the plurality of shafts 115 with the control interface 220. For example, an outer sheath clutch 334 selectively couples the outer sheath 120 with the control interface 220. An outer shaft fitting 332 is coupled with the outer shaft 130; and an inner shaft clutch is coupled with the inner shaft 140. With engagement of one or more of the clutches 330 (334 and inner sheath clutch) the control interface 220 is engaged with the corresponding component (e.g., shafts 120, 130, 140) and operable to actuate the component(s). When one or more plurality of clutches 330 are engaged, the corresponding shaft (or shafts) of the plurality of shafts 115 are movable with actuation of the control interface 220. Movement of the control interface 220 is transmitted to the components having the clutches engaged (directly or indirectly) with the interface 220. For instance, movement of the control interface 220 with the hand or thumb of the technician correspondingly moves one or more of the outer sheath 120, the outer shaft 130 or the inner shaft 140 (e.g., alone or in combination depending on engagement of the respective clutches). For example, each of the plurality of clutches 330 transmits a corresponding motion and relative movement to the associated shaft of the plurality of shafts 115. For example, the control interface 220 transmits a corresponding rotation to the associated shaft of the plurality of shafts 115 and the associated shaft transmits rotation to the cage 150. In other examples, the hub 210 is rotated to translate rotation to the associated shaft of the plurality of shafts and the associated shaft transmits rotation to the cage 150.In operation, as illustrated in FIGS. 4 and 5A-5D, the catheter system 100 (See FIG. 1) is an extraction device that is operated to remove a blockage, such as a blood clot or thrombus, from a body lumen. A medical professional actuates the catheter system 100 to advance the plurality of shafts 115 through selected bodily lumens to the target location of the blockage. Once the distal end 112 of the plurality of shafts 115 is at or proximate to the blockage the medical professional engages the control mechanism 200.

In an example illustrated in FIGS. 5A-5D , the medical professional manipulates (e.g., shifts, moves, rotates or the like) the control interface 220 in a proximal direction from a first position 410 (originating position) (FIG. 5A) along a first segment of the body hub to a second position 412 (FIG. 5B). The control interface 220 movement is constrained with a restrictor 320 within a guide, groove, protrusion, recess (hereinafter “guide” 338) or the like, on a shuttle 336 within the hub 210 of the control mechanism 200, as illustrated in FIG. 3. In an example, the control interface 220 is coupled with an outer sheath clutch 334 and the outer sheath clutch 334 is coupled with the outer sheath 120. As illustrated in FIG. 3, the outer sheath clutch 334 engages with the shuttle 336 when the outer sheath clutch 334 is translated (as illustrated in 5B) in the proximal direction with the proximal movement of the control interface 220. The movement of the control interface 220 from the first position 410 to the second position 412 is transmitted to the outer sheath clutch 334 and then the outer sheath 120 to slide or otherwise move the outer sheath 120 in a proximal direction. When the outer sheath 120 is withdrawn proximally the collapsed cage 150 is exposed.

In an example, when the outer sheath 120 is moved in a proximal direction (e.g., retracted) the collapsed cage 150 is exposed. The control interface 220 then disengages from the outer sheath clutch 334. The control interface 220 is then moved in a proximal direction along a second segment of the hub body and translated further to a second position in the proximal direction with movement of the control interface 220. For example, the shuttle 336 moves over the outer shaft fitting 332 in a continued movement, as the outer shaft fitting 332 remains in a fixed position within the hub 210. In this configuration, the outer shaft fitting 332 retains the outer shaft 130 in a fixed position.

In an example as illustrated in FIG. 5C, after the outer sheath 120 is moved in a proximal direction (e.g., retracted) and the cage 150 is uncovered, the cage 150 is then expanded or opened. For example, the proximal portion 154 of the cage 150 is coupled with a distal portion 132 of the outer shaft 130 and the distal portion 152 of the cage 150 is coupled with a distal portion 142 of the inner shaft 140. The cage 150 is expanded, or opened to a cage configuration, by further manipulating the control interface 220 in a continued proximal direction. The continued proximal direction is, for example, a direction that is similar, collinear or corresponds with the first movement in the proximal direction.

When the control interface 220 is moved in a continued proximal direction to be engaged with the inner shaft clutch, or the proximal end 339 of the track 338 on the shuttle 336 (see FIG. 3). When the inner shaft clutch is translated in a proximal direction, the inner shaft 140 is translated in a proximal direction. With the distal portion 152 of the cage 150 coupled with the distal portion 142 of the inner shaft 140 and the proximal portion 154 of the cage with the distal portion 132 of the outer shaft 130, movement of the inner shaft 140 in the proximal direction pulls, or similarly moving, the distal portion 152 of cage 150 towards the proximal portion 154 of the cage 150. Pulling the distal portion 152 towards the proximal portion 154 of the cage 150 causes the diameter of the cage 150 to expand.

The control interface 220, in an example, continues to move in a proximal direction until resistance is met. For example, the resistance is met when the inner catheter clutch 205, or the shuttle 336 abuts a control spring 207 within the control mechanism 200. When the control interface 220 reaches the point of resistance, the inner catheter clutch 205 is mechanically released from engagement and the control interface 220 is controlled by biasing forces exerted against the inner catheter clutch or shuttle 336, via the control spring 207. The cooperation between the control interface 220, the inner catheter clutch 205 or shuttle 336 and the cage 150, and the control spring 207 allows the cage 150 to expand and contract according to the contours of the bodily lumen. For example, the control spring 207 biases the cage in an open configuration. In an example, the cage 150 remains in an expanded configuration but not in a configuration where the cage 150 can be damaged, clogged or otherwise during use.

In an example, the cage 150 remains expanded during use, to draw the blockage through openings 155 in the cage 150. For example, the control interface 220 maintains the expansion of the cage 150 during use. For example, cage 150 has a profile suitable for scraping the inner surfaces of a bodily lumen or otherwise engaging with the blockage to draw the blockage through openings 155 in the cage 150 and towards the inner shaft 140. In examples, the control interface 220 transmits rotation to the cage 150 to scrape the inner surfaces of a body lumen. In other examples, the hub 210 is rotated to translate rotation to the cage 150. Optionally, the window 165 in the inner shaft 140 provides access for drawing the blockage towards the extractor 160 housed or contained within the inner shaft 140. The extractor 160 transports the blockage through the inner shaft 140 and out of the catheter system 100.

The design of the catheter system 100 provides for the process of removing the blockage to be repeated. For example, as the cage 150 is pulled in a proximal direction during removal of the blockage, not all of the blockage may be collected. If not all of the blockage is collected, the cage 150 needs to be removed from the bodily lumen before readvancing to through the blockage. In another example, the cage 150 needs to move in a distal direction to re-capture or collect remaining fragments of the blockage. In either example where there cage 150 is withdrawn from the body or where the cage 150 is advanced in the distal direction, the cage 150 is in a closed, or sheathed, configuration as the cage 150 passes through the bodily lumen. If the cage needs to pass back through the blockage, the outer sheath 120 is pulled over the cage by reversing the movement of the control interface 220.

As illustrated in FIG. 5D, reversing the direction of the control interface 220, the control interface 220 is moved in a distal direction thereby moving the inner shaft in a distal direction. Moving the inner shaft 140 in a distal direction causes the cage to collapse by moving the distal end of the cage in the distal direction.

As further illustrated in FIG. 5D, and in an example, further movement of the control interface 220 in the distal direction translates the outer sheath 120 to move in the distal direction. Moving the outer sheath 120 in the distal direction advances the outer sheath 120 over the cage 150. In an example, when the outer sheath 120 is moved over the cage 150 after the cage 150 has been used, remnants of the blockage can be retained within the cage 150. Excess stress or strain is placed on the plurality of shafts 115 when remnants remain. The strain relief 240 (illustrated in FIG. 2A) provides an example of a way to minimize damage to the plurality of shafts 115 in a situation where the medical professional needs to provide more force to the control interface 220 to advance the outer sheath 120 over the cage 150. Re-sheathing the cage 150 allows the plurality of shafts 115 to either be advanced to a target location on a distal side of the blockage or be withdrawn in a proximal direction through the body lumens.

### Various Notes and Aspects

Aspect 1 can include subject matter such as a catheter system comprising: a catheter shaft including: an outer sheath; an outer catheter shaft within the outer sheath; and an inner catheter shaft movably coupled within the outer catheter shaft; a cage proximate to a distal end of the catheter shaft having a distal portion of the cage coupled with the inner catheter shaft and a proximal portion of the cage coupled with the outer catheter shaft; and a hub assembly coupled with the catheter shaft, the hub assembly including: a hub body; and a control interface movably coupled with the hub body and configured to actuate the outer sheath and the cage, and the control interface has respective sheath and cage configurations: in the sheath configuration, the control interface is moved with a first movement in an opening direction to retract the outer sheath relative to the cage; and in the cage configuration, the control interface is moved with a second movement in the opening direction to deploy the cage.

Aspect 2 can include, or can optionally be combined with the subject matter of Aspect 1, to optionally include wherein the first movement corresponds with the second movements in the first direction.

Aspect 3 can include, or can optionally be combined with the subject matter of one or any combination of Aspects 1 or 2 to optionally include wherein the first movement and second movement are collinear in the opening direction.

Aspect 4 can include, or can optionally be combined with the subject matter of one or any combination of Aspects 1-3 to optionally include wherein the control interface has an engageable surface, and the control interface is configured to move with the first movement from an originating position to at least a second position.

Aspect 5 can include, or can optionally be combined with the subject matter of one or any combination of Aspects 1-4 to optionally include wherein the control interface is configured to move with the second movement from the second position to at least a third position; wherein the first movement and the second movement are in a proximal direction of the catheter system.

Aspect 6 can include, or can optionally be combined with the subject matter of Aspects 1-5 to optionally include wherein the first movement is in a proximal direction along a first segment of the hub body, and the second movement is in a proximal direction along a second segment of the hub body extending from the first segment.

Aspect 7 can include, or can optionally be combined with the subject matter of Aspects 1-6 to optionally include wherein a plurality of clutches are housed in the hub and each of the plurality of clutches are selectively coupled with the control interface.

Aspect 8 can include, or can optionally be combined with the subject matter of Aspects 1-7 to optionally include wherein at least one of the plurality of clutches is coupled with at least one of the inner catheter shaft, the outer catheter shaft or the outer sheath.

Aspect 9 can include, or can optionally be combined with the subject matter of Aspects 1-8 to optionally include a biasing spring housed within the hub; and wherein the biasing spring is engageable with at least one of the plurality of clutches; wherein the biasing spring is configured to bias the cage in an open configuration.

Aspect 10 can include, or can optionally be combined with the subject matter of Aspects 1-9 to optionally include wherein the outer catheter shaft is in a fixed configuration.

Aspect 11 can include, or can optionally be combined with the subject matter of Aspects 1-10 to optionally include wherein the outer sheath is configured to encase at least the distal end of the cage, the inner catheter shaft and the outer catheter shaft.

Aspect 12 can include, or can optionally be combined with the subject matter of Aspects 1-11 to optionally include wherein a first clutch is coupled with the outer sheath and a second clutch is coupled with the inner catheter shaft.

Aspect 13 can include, or can optionally be combined with the subject matter of Aspects 1-12 to optionally include an extraction device, comprising: a catheter shaft including: an outer sheath having a sheath clutch; an outer catheter shaft within the sheath; and an inner catheter shaft movably coupled with respect to the outer catheter shaft, the inner catheter shaft having a shaft clutch; a rotatable extractor coil extending within the inner catheter shaft; a cage having a distal end coupled with a distal end of the inner catheter shaft and a proximal end of the cage coupled with a distal end of the outer catheter shaft; and a control system coupled with the catheter shaft, the control system including a control interface, the control interface having a engageable mechanism; wherein the control interface is selectively couplable with each of the sheath clutch and shaft clutch; wherein the engageable mechanism is configured to translate movement to the sheath clutch and the shaft clutch.

Aspect 14 can include, or can optionally be combined with the subject matter of Aspects 1-13 to optionally include wherein the control system is configured to transfer a first movement from the control interface to the outer sheath in a sheath configuration and to transfer a second movement from the control interface to the inner catheter shaft in a cage configuration.

Aspect 15 can include, or can optionally be combined with the subject matter of Aspects 1-14 to optionally include wherein in the cage configuration, the second movement expands the cage.

Aspect 16 can include, or can optionally be combined with the subject matter of Aspects 1-15 to optionally include wherein the control interface is configured to translate the outer sheath in a proximal direction and a distal direction.

Aspect 17 can include, or can optionally be combined with the subject matter of Aspects 1-16 to optionally include wherein the control system includes a control spring; wherein when the control spring is engaged, the control spring biases the cage in an open configuration.

Aspect 18 can include, or can optionally be combined with the subject matter of Aspects 1-17 to optionally include wherein the control interface is a thumb slider configured to move in a proximal or distal direction and configured to be pressed in a direction transverse to the proximal or distal direction.

Aspect 19 can include, or can optionally be combined with the subject matter of Aspects 1-18 to optionally include a method of operating a catheter system comprising: engaging a control interface of a control mechanism; wherein the control mechanism is coupled with a hub; wherein the control mechanism includes: a first clutch configured to movably couple the control mechanism with an outer catheter sheath; a second clutch configured to movably couple the control mechanism with an inner catheter shaft; wherein the inner catheter shaft is movable coupled within the outer catheter shaft; shifting the position of the control interface from an originating position to a second position; wherein shifting the position of the control interface from the originating position to the second position translates movement from the control interface to the first clutch; wherein the first clutch moves the outer sheath in a proximal direction and exposes a cage; wherein a distal portion of the cage is coupled with the inner catheter shaft and a proximal portion of the cage coupled with the outer catheter shaft; shifting the position of the control interface from the second position to a third position; wherein shifting the position of the control interface from the second position to the third position translates movement from the control interface to the second clutch; wherein the second clutch translates the inner catheter shaft in a proximal direction; wherein translating the inner catheter shaft in a proximal direction expands the cage coupled at a proximal end to an outer catheter shaft and coupled at a distal end to the inner catheter shaft; maintaining the control interface in the third position to maintain expansion of the cage; moving the control interface in a distal direction from the third position to the second position to collapse the cage; and moving the control interface from the second position to the originating position to move the sheath in the distal direction to substantially cover the cage.

Aspect 20 can include, or can optionally be combined with the subject matter of Aspects 1-19 to optionally include wherein the outer catheter shaft remains substantially static when moving the inner catheter shaft.

Aspect 21 can include, or can optionally be combined with the subject matter of Aspects 1-20 to optionally include locking the control interface in the third position by applying a force to the control interface; and applying a force to the control interface to unlock the control interface.

Aspect 22 can include, or can optionally be combined with the subject matter of Aspects 1-21 to optionally include wherein a biasing member engages with the second clutch to substantially maintain the cage in an open configuration.

Aspect 23 can include, or can optionally be combined with the subject matter of Aspects 1-22 to optionally include activating an extractor coil housed within the inner catheter shaft when the cage is in an open configuration.

Each of these non-limiting aspects can stand on its own, or can be combined in various permutations or combinations with one or more of the other aspects.

The above description includes references to the accompanying drawings, which form a part of the detailed description. The drawings show, by way of illustration, specific embodiments in which the invention can be practiced. These embodiments are also referred to herein as “aspects” or “examples.” Such aspects or example can include elements in addition to those shown or described. However, the present inventors also contemplate aspects or examples in which only those elements shown or described are provided. Moreover, the present inventors also contemplate aspects or examples using any combination or permutation of those elements shown or described (or one or more features thereof), either with respect to a particular aspects or examples (or one or more features thereof), or with respect to other Aspects (or one or more features thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

The above description is intended to be illustrative, and not restrictive. For example, the above-described aspects or examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as aspects, examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A catheter system comprising:
a catheter shaft including:
an outer sheath;
an outer catheter shaft within the outer sheath; and
an inner catheter shaft movably coupled within the outer catheter shaft;
a cage proximate to a distal end of the catheter shaft having a distal portion of the cage coupled with the inner catheter shaft and a proximal portion of the cage coupled with the outer catheter shaft; and
a hub assembly coupled with the catheter shaft, the hub assembly including:
a hub body; and
a control interface movably coupled with the hub body and configured to actuate the outer sheath and the cage, and the control interface has respective sheath and cage configurations:
in the sheath configuration, the control interface is moved with a first movement in an opening direction to retract the outer sheath relative to the cage; and
in the cage configuration, the control interface is moved with a second movement in the opening direction to deploy the cage.

2. The catheter system of claim 1, wherein the first movement corresponds with the second movements in the first direction.

3. The catheter system of claim 2, wherein the first movement and second movement are collinear in the opening direction.

4. The catheter system of claim 1, wherein the control interface has an engageable surface, and the control interface is configured to move with the first movement from an originating position to at least a second position.

5. The catheter system of claim 4, wherein the control interface is configured to move with the second movement from the second position to at least a third position;
wherein the first movement and the second movement are in a proximal direction of the catheter system.

6. The catheter system of claim 1, wherein the first movement is in a proximal direction along a first segment of the hub body, and the second movement is in a proximal direction along a second segment of the hub body extending from the first segment.

7. The catheter system of claim 1, wherein a plurality of clutches are housed in the hub and each of the plurality of clutches are selectively coupled with the control interface.

8. The catheter system of claim 7, wherein at least one of the plurality of clutches is coupled with at least one of the inner catheter shaft, the outer catheter shaft or the outer sheath.

9. The catheter system of claim 8, further comprising a biasing spring housed within the hub; and
wherein the biasing spring is engageable with at least one of the plurality of clutches;
wherein the biasing spring is configured to bias the cage in an open configuration.

10. The catheter system of claim 1, wherein the outer catheter shaft is in a fixed configuration.

11. The catheter system of claim 1, wherein the outer sheath is configured to encase at least the distal end of the cage, the inner catheter shaft and the outer catheter shaft.

12. The catheter system of claim 1, wherein a first clutch is coupled with the outer sheath and a second clutch is coupled with the inner catheter shaft.

13. An extraction device, comprising:
a catheter shaft including:
an outer sheath having a sheath clutch;
an outer catheter shaft within the sheath; and
an inner catheter shaft movably coupled with respect to the outer catheter shaft, the inner catheter shaft having a shaft clutch;
a rotatable extractor coil extending within the inner catheter shaft;
a cage having a distal end coupled with a distal end of the inner catheter shaft and a proximal end of the cage coupled with a distal end of the outer catheter shaft; and
a control system coupled with the catheter shaft, the control system including a control interface, the control interface having an engageable mechanism;
wherein the control interface is selectively couplable with each of the sheath clutch and shaft clutch;
wherein the engageable mechanism is configured to translate movement to the sheath clutch and the shaft clutch.

14. The extraction device of claim 13, wherein the control system is configured to transfer a first movement from the control interface to the outer sheath in a sheath configuration and to transfer a second movement from the control interface to the inner catheter shaft in a cage configuration.

15. The extraction device of claim 14, wherein in the cage configuration, the second movement expands the cage.

16. The extraction device of claim 13, wherein the control interface is configured to translate the outer sheath in a proximal direction and a distal direction.

17. The extraction device of claim 13 wherein the control system includes a control spring;
wherein when the control spring is engaged, the control spring biases the cage in an open configuration.

18. The extraction device of claim 13 wherein the control interface is a thumb slider configured to move in a proximal or distal direction and configured to be pressed in a direction transverse to the proximal or distal direction.

19. A method of operating a catheter system comprising:
engaging a control interface of a control mechanism;
wherein the control mechanism is coupled with a hub;
wherein the control mechanism includes:
a first clutch configured to movably couple the control mechanism with an outer catheter sheath;
a second clutch configured to movably couple the control mechanism with an inner catheter shaft;
wherein the inner catheter shaft is movable
coupled within the outer catheter shaft;
shifting the position of the control interface from an originating position to a second position;
wherein shifting the position of the control interface from the originating position to the second position translates movement from the control interface to the first clutch;
wherein the first clutch moves the outer sheath in a proximal direction and exposes a cage;
wherein a distal portion of the cage is coupled with the inner catheter shaft and a proximal portion of the cage coupled with the outer catheter shaft;
shifting the position of the control interface from the second position to a third position;
wherein shifting the position of the control interface from the second position to the third position translates movement from the control interface to the second clutch;
wherein the second clutch translates the inner catheter shaft in a proximal direction;
wherein translating the inner catheter shaft in a proximal direction expands the cage coupled at a proximal end to an outer catheter shaft and coupled at a distal end to the inner catheter shaft;
maintaining the control interface in the third position to maintain expansion of the cage;
moving the control interface in a distal direction from the third position to the second position to collapse the cage; and
moving the control interface from the second position to the originating position to move the sheath in the distal direction to substantially cover the cage.

20. The method of operating the catheter system of claim 19, wherein the outer catheter shaft remains substantially static when moving the inner catheter shaft.

21. The method of operating the catheter system of claim 20, further comprising:
locking the control interface in the third position by applying a force to the control interface; and
applying a force to the control interface to unlock the control interface.

22. The method of operating the catheter system of claim 19, wherein a biasing member engages with the second clutch to substantially maintain the cage in an open configuration.

23. The method of operating the catheter system of claim 19, further comprising:
activating an extractor coil housed within the inner catheter shaft when the cage is in an open configuration.
